(19) United States
(12) Patent Application Publication
(10) Pub. No.: US 2018/0153899 A1
(43) Pub. Date: Jun. 7, 2018

(54) PHARMACEUTICAL COMPOSITIONS

(71) Applicant: Novartis AG, Basel (CH)

(72) Inventors: Sarah Elizabeth DAVID, Bottmingen (CH); Christian KAMINSKI, Grenzach-Wyhlen (DE)

(21) Appl. No.: 15/576,001

(22) PCT Filed: May 19, 2016
(86) PCT No.: PCT/IB2016/052943

§ 371 Date: Nov. 21, 2017

Related U.S. Application Data
(60) Provisional application No. 62/165,333, filed on May 22, 2015.

(51) Int. Cl. A61K 31/55; A61K 9/20; A61K 9/48; A61K 9/28

(52) U.S. Cl. CPC A61K 31/55; A61K 9/2018; A61K 9/4858; A61K 9/2054; A61K 9/4866; A61K 9/2027; A61K 9/4833; A61K 9/2866; A61K 9/2009; A61K 9/485; A61K 9/2095; A61K 9/2893; A61K 9/2013

(57) ABSTRACT

Pharmaceutical compositions comprising the drug substance (R,E)-N-(7-chloro-1-(1-(4-(dimethylamino)but-2-enoyl)azepan-3-yl)-1H-benzo[d]imidazol-2-yl)-2-methylisonicotinamide and processes to prepare these compositions.

### FIELD OF THE INVENTION

The present invention relates to pharmaceutical compositions comprising the drug substance (R,E)-N-(7-chloro-1-(1-(4-(dimethylamino)but-2-enoyl)azepan-3-yl)-1H-benzo[d]imidazol-2-yl)-2-methylisonicotinamide, and processes to prepare said pharmaceutical compositions.

### BACKGROUND OF THE INVENTION

The drug substance (R,E)-N-(7-chloro-1-(1-(4-(dimethylamino)but-2-enoyl)azepan-3-yl)-1H-benzo[d]imidazol-2-yl)-2-methylisonicotinamide, also referred to as EGF816, and herein also referred to as compound of formula (1),

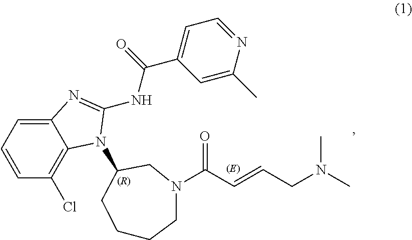

was found to act as epidermal growth factor receptor (EGFR) antagonist and useful for the treatment of non-small cell lung cancer (NSCLC). References: “EGF816, a novel covalent inhibitor of mutant-selective epidermal growth factor receptor, overcomes T790M-mediated resistance in NSCLC,” American Association for Cancer Research Annual Meeting, Jie Li, et al., Vol 105th, Issue Apr. 7, 2014; and “In vitro characterization of EGF816, a third-generation mutant-selective EGFR inhibitor,” American Association for Cancer Research Annual Meeting, Yong Jia, et al., Vol 105th, Issue Apr. 7, 2014. The content of said two references is incorporated herein by reference.

The chemical synthesis to prepare said drug substance was described in WO2013/184757, the content of which is incorporated herein by reference. Further, various crystalline forms of said drug substance were described in PCT/CN2013/088295, the content of which is incorporated herein by reference.

### SUMMARY OF THE INVENTION

Because every drug substance (DS), also referred to as active pharmaceutical ingredient (API), has its own physical, chemical and pharmacological characteristics a pharmaceutical composition has to be individually designed for every new API.

The design of a pharmaceutical composition for drug substance compound of formula (1) is especially difficult for (inter alia) the following reasons:

The compound contains amine groups which are prone to undergo undesired chemical reactions by attacking as electrophiles nucleophilic centers of other components of the composition, e.g. carbonyl units of aldehydes or esters. Some pharmaceutical excipients may therefore turn out to be incompatible with the compound. The compound contains further a double bond which is also prone to be subject of undesired chemical reactions.

Especially in its mesylate trihydrate form, the compound in solid form is very cohesive and shows poor flowability which makes pharmaceutical processing difficult. The compound was observed to show a strong tendency to adhere to metal surfaces of pharmaceutical processing equipment, e.g. tabletting dies and punches, causing stickiness issues. Further, as trihydrate, the compound may undergo undesired crystalline form conversions when the pharmaceutical processing involves steps such as wetting and drying.

In trials to design a pharmaceutical composition for the compound of formula (1), it was found that many pharmaceutical excipients negatively influence the dissolution rate of the drug substance from a solid dosage form such as a tablet. For example, the disintegrant croscarmellose sodium (NaCMC-XL) was found to interact with the compound causing incomplete drug dissolution (only ca. 70% after 60 min at pH 6.8). As further example, low-substituted hydroxypropylcellulose (L-HPC) as disintegrant or dicalciumphospate (DCP) as filler were found to slow down drug dissolution rate. Other excipients, such as sodium starch glycolate (SSG) as disintegrant, which were not hindering drug dissolution, were found to show in compositions with the compound of formula (1) poor compressibility or microcrystalline cellulose (MCC) as sole filler, caused a strong negative impact on tablet disintegration.

In view of the above mentioned difficulties, it was very uncertain whether a pharmaceutical composition for the compound of formula (1) could be designed which allows the manufacturing of an oral dosage form, e.g. a tablet, on a commercial scale and in the high quality which is required for human medicines and in a quality that provides for commercially reasonable shelf-life. However, it was surprisingly found that the application of two specific fillers together with a specific distintegrant resulted in a stable and pharmaceutically processible composition of good compression properties, and at the same time of fast dissolution, as well as fast disintegration characteristics.

Therefore, in a first aspect of the present invention, there is provided a pharmaceutical composition comprising
(a) the drug substance (R,E)-N-(7-chloro-1-(1-(4-(dimethylamino)but-2-enoyl)azepan-3-yl)-1H-benzo[d]imidazol-2-yl)-2-methylisonicotinamide, a pharmaceutically acceptable salt, hydrate, or salt hydrate thereof,
(b) the fillers mannitol and microcrystalline cellulose,
(c) the disintegrant crospovidone, and
(d) a lubricant.

In a second aspect of the invention, there is provided a process for the preparation of a pharmaceutical composition as defined by the first aspect comprising the following steps:
(1) dry granulation of a blend composed of
   (a) the drug substance (R,E)-N-(7-chloro-1-(1-(4-(dimethylamino)but-2-enoyl)azepan-3-yl)-1H-benzo[d]imidazol-2-yl)-2-methylisonicotinamide, a pharmaceutically acceptable salt, hydrate, or salt hydrate thereof, preferably the mono-mesylate trihydrate salt thereof,
   (b) the filler microcrystalline cellulose, preferably of the quality 101,
   (c) the disintegrant crospovidone,
   (d) a lubricant, preferably magnesium stearate, and
   optionally (e) the filler mannitol, and
   optionally (f) a glidant, preferably colloidal silicon dioxide,
   to obtain granules;
(2) compression of the granules obtained by step (1) together with a blend composed of
   (g) the filler microcrystalline cellulose, preferably of the quality 102,
   (h) the disintegrant crospovidone,
   (i) a lubricant, preferably magnesium stearate, and
   optionally (j) the filler mannitol, and
   optionally (k) a glidant, preferably colloidal silicon dioxide,
   to obtain tablets;
wherein in either step (1) or step (2) the filler mannitol (component (e) or (j)) must be used;
and optionally
(3) film coating of the tablets obtained by step (2), preferably with coating suspension or solution composed of hypromellose.

### BRIEF DESCRIPTION OF THE DRAWINGS

In the following the present invention is described in detail with reference to accompanying figures in which:

FIG. 1 shows the dissolution rate curves for the test batch compositions of example 4: test batch 4-1 (“SSG”, squares), test batch 4-2 (“PVP-XL”, circles), test batch 4-3 (“L-HPC”, triangles). The figure demonstrates that the order of dissolution rate is PVP-XL>SSG>L-HPC.

FIG. 2 shows the hardness versus compression force curves for the test batch compositions of example 4: test batch 4-1 (“SSG”, squares), test batch 4-2 (“PVP-XL”, circles), test batch 4-3 (“L-HPC”, triangles). The figure demonstrates that the order of compressibility is L-HPC>PVP-XL>SSG.

FIG. 3 shows the tensile strength versus compression force curves for the test batch compositions of example 4: test batch 4-1 (“SSG”, squares), test batch 4-2 (“PVP-XL”, circles), test batch 4-3 (“L-HPC”, triangles). The figure demonstrates that the order of compressibility is L-HPC>PVP-XL>SSG.

### DETAILED DESCRIPTION OF THE INVENTION

Herein after, the present invention is described in further detail and is exemplified.

In all aspects of the invention, the active pharmaceutical ingredient (API) or drug substance (DS) is (R,E)-N-(7-chloro-1-(1-(4-(dimethylamino)but-2-enoyl)azepan-3-yl)-1H-benzo[d]imidazol-2-yl)-2-methylisonicotinamide, a pharmaceutically acceptable salt, hydrate, or salt hydrate thereof.

For example, the API may be the free form (i.e. not a salt) in an amorphous or crystalline state. Said free forms may be anhydrous or present as hydrate. Alternatively, the API may be a salt in an amorphous of crystalline state. Said salt may be anhydrous or present as hydrate.

Preferably, in the aspects of the invention, the API is present as mesylate (methylsulphonate) salt, more preferably as mono-mesylate salt. Said mesylate salts may be in an amorphous of crystalline state. Preferably, said mesylate salts are in a crystalline state.

More preferably, said mesylate salts are present as hydrates, e.g. monohydrate, dihydrate or trihydrate. Said mesylate salt hydrates may be amorphous or crystalline. Even more preferably, the API in the aspects of the present invention is the mono-mesylate salt trihydrate in crystalline form. Most preferably, the API is the crystalline mesylate trihydrate form B as described in PCT/CN2013/088295, example 3, and has the following characteristic x-ray powder diffraction pattern (XRPD): 11.76, 13.832, 14.41, 15.9 17.65, 18.79, 21.46, 21.83, 22.30, 23.82, 24.51, 24.89, 25.57, 26.66 and 27.77±0.30 2θ (CuKα λ=1.54056 Å). It may be characterized by a XRPD comprising five or more 20 values (CuKα λ=1.54056 Å) selected from the group consisting of 11.76, 13.832, 14.41, 15.9 17.65, 18.79, 21.46, 21.83, 22.30, 23.82, 24.51, 24.89, 25.57, 26.66 and 27.77±0.30, measured at a temperature of about 22° C. Preferably, said form B may be characterized by an x-ray powder diffraction pattern comprising six or more 26 values (CuKα λ=1.54056 Å) selected from the group consisting of 11.76, 13.832, 14.41, 15.9 17.65, 18.79, 21.46, 21.83, 22.30, 23.82, 24.51, 24.89, 25.57, 26.66 and 27.77±0.30, at a temperature of about 22° C.

In accordance with the first aspect of the present invention, there is provided a pharmaceutical composition comprising
(a) the drug substance (R,E)-N-(7-chloro-1-(1-(4-(dimethylamino)but-2-enoyl)azepan-3-yl)-1H-benzo[d]imidazol-2-yl)-2-methylisonicotinamide, a pharmaceutically acceptable salt, hydrate, or salt hydrate thereof,
(b) the fillers mannitol and microcrystalline cellulose,
(c) the disintegrant crospovidone, and
(d) a lubricant.

Compositions with microcrystalline cellulose (MCC) showed good compressibility and formed ribbons of good quality in roller compactors. MCC contributed to the avoidance of sticking issues. However, compositions with MCC alone did not disintegrate well and required a further filler.

Mannitol was found to be a suitable further filler as it contributed to the avoidance of sticking issues and facilitated disintegration.

Other fillers were associated with disadvantages. Compositions with dicalciumphosphate showed only slow drug release and tablets made with compositions containing lactose were affected by capping issues. Further, lactose as reducing sugar bears the risk of chemical instabilities with the drug substance of the present invention.

The disintegrant crospovidone was found to provide good compression and at the same time ensures fast dissolution. Other disintegrants were associated with disadvantages. Compositions with L-HPC showed only slow dissolution. Those with sodium starch glycolate (SSG) showed fast drug release but were found to be poorly compressible. Compositions with croscarmellose (CMC-XL, e.g. Ac-Di-Sol by FMC BioPolymer) showed release of only less than 100% in pH 4.5-6.8 and caused physical incompatibilities (excipient-drug absorption effects).

Preferably, in said pharmaceutical composition said drug substance is present as mesylate (methylsulphonate) salt, preferably as mono-mesylate salt, more preferably as mono-mesylate trihydrate salt.

Preferably, in said pharmaceutical compositions, said drug substance, calculated based on its free base and on an anhydrous basis (salt former and water not considered in this calculation), is present from 5 to 50%, more preferably from 10 to 40%, even more preferably from 20 to 30% by weight based on the total weight of said pharmaceutical composition. This high amount of drug load ensures that for high doses the tablet remains swallowable.

Preferably, in said pharmaceutical compositions, said fillers together are present from 20 to 90%, more preferably 50 to 70%, even more preferably 55 to 65% by weight based on the total weight of said pharmaceutical composition.

Preferably, in said pharmaceutical compositions, said fillers are mannitol and microcrystalline cellulose, present in a ratio of from 3:1 to 1:1, more preferably from 2.5:1.0 to 1.5:1.0, even more preferably from 2.2:1.0 to 1.8:1.0, most preferably about 2:1 (weight of mannitol:weight of microcrystalline cellulose).

Preferably, in said pharmaceutical compositions, the filler mannitol (Ph.Eur., USP-NF) or D-mannitol (JP) is of a quality suitable for direct compression (mannitol DC), e.g. spray-dried or granulated mannitol which is available e.g. from Roquette under the trade name Pearlitol. Said granulated mannitol may have a mean diameter of from 200 to 600 micrometer, preferably 250 to 520 micrometer.

Preferably, in said pharmaceutical compositions, the filler microcrystalline cellulose (Ph.Eur., USP-NF, JP) is of a quality selected from 101 and 102, e.g. Avicel® PH101 (nominal mean particle size 50 micrometer, Particle size analysis: mesh size 60, amount retained≤1.0%, mesh size 200, amount retained≤30.0%) and Avicel®PH102 (nominal mean particle size 100 micrometer, particle size analysis: mesh size 60, amount retained ≤8.0%, mesh size 200, amount retained≥45.0%) available by FMC BioPolymer or Vivapur®101 (particle size from 45 to 80 micrometer) and Vivapur®102 (particle size from 90 to 150 micrometer) available by JRS Pharma (JRS=J. Rettenmaier & Sohne).

The disintegrant crospovidone (Ph.Eur., USP-NF, JP), used in said pharmaceutical compositions, may be of the quality Ph.Eur. crospovidone monograph type A or type B. Preferably, the quality is type A. Preferably, this type A quality has an average particle size from 110 to 140 microns and peroxides to a maximum of 400 ppm. More preferably, the quality of the crospovidone is equivalent to the quality available under the trade name Polyplasdone XL from Ashland in the grade “XL”.

Preferably, in said pharmaceutical compositions, the disintegrant is present from 2 to 10%, more preferably 3 to 8%, even more preferably 4 to 7% by weight based on the total weight of said pharmaceutical composition.

Said pharmaceutical compositions contain one or more lubricants.

The term “lubricants” refers herein to those pharmaceutical excipients which have the primary function of decreasing friction at the interface between a tablet's surface and the die wall during ejection and of reducing wear on punches and dies and of preventing sticking to punch faces or in the case of encapsulation of preventing sticking to machine dosators, tamping pins, etc.

Lubricants may be selected from the group of fatty acids or their salts, e.g. stearic acid or any of its salts (e.g. calcium, zinc, or magnesium stearate), lauryl sulfuric acid or any of its salts (e.g. sodium or magnesium lauryl sulfate), stearyl fumaric acid or any of its salts (e.g. sodium stearyl fumarate), fatty acid esters, e.g. Glyceryl dibehenate (Compritol® 888 ATO), polyethylene glycol, and liquid paraffin.

Preferably, in said pharmaceutical compositions, the lubricant is a stearic acid or any of its metal salts, more preferably said lubricant is calcium or magnesium stearate, even more preferably said lubricant is magnesium stearate (Ph.Eur., USP-NF, JP).

Preferably, in said pharmaceutical compositions, said lubricant is present in from 1 to 5%, preferably 2 to 4%, more preferably 2 to 3% by weight based on the total weight of said pharmaceutical composition. The unusually large amount of lubricant is important to overcome the strong sticking issues associated with the drug substance of the present invention. This high amount of hydrophobic lubricant usually causes slower dissolution and disintegration. However, it was surprisingly found that the pharmaceutical compositions of the present invention were still able to provide fast drug dissolution and quick disintegration. Mannitol and crospovidone were found to provide in the compositions of the present invention, a positive effect with respect to facilitating drug dissolution and disintegration.

Said pharmaceutical composition may contain a glidant.

The term “glidant” refers herein to those pharmaceutical excipients which have the primary function of enhancing product flow by reducing interparticulate friction.

The glidant may be selected from the group of silaceous material, e.g. syloid, pyrogenic silica, hydrated sodium siliosluminate, and talc.

Preferably, the pharmaceutical compositions of the present invention comprise a glidant, said glidant is preferably a colloidal silicon dioxide (USP-NF) (also referred to as colloidal anhydrous silica (BP), light anhydrous silicic acid (JP), silica, colloidal anhydrous (Eu.Phr.)), preferably with a specific surface area of 200±25 m²/g, e.g. AEROSIL 200 by Evonik Industries.

The pharmaceutical compositions of the present invention may be in the pharmaceutical dosage form of a powder, capsule, or tablet, preferably a tablet.

Preferably, said tablet is coated with a film, preferably said film comprises hypromellose, e.g. by using a coating premix composition, e.g. Opadry I by Colorcon (containing hypromellose, polyethylene glycol (PEG) 4000, talc as well as a colorant, e.g. iron oxide, read or black, titanium dioxide). Preferaly, Opadry I by Colorcon is used.

Said pharmaceutical dosage form may comprise a drug substance dose selected from 10, 25, 50, 75, 100, 150, and 200 mg, preferably the dose is selected from 25, 50, 75, and 100 mg, more preferably the dose is 50 mg of the drug substance referred to as its free base and in its anhydrous form.

In one embodiment of the present invention, the pharmaceutical composition comprises:
(a) 5-50% by weight of the drug substance (R,E)-N-(7-chloro-1-(1-(4-(dimethylamino)but-2-enoyl)azepan-3-yl)-1H-benzo[d]imidazol-2-yl)-2-methylisonicotinamide, calculated based on its free base and on its anhydrous basis, present as mono-mesylate trihydrate salt,
(b) 20-90% by weight of the fillers of mannitol and microcrystalline cellulose together,
(c) 2-10% by weight of the disintegrant crospovidone,
(d) 1-5% by weight of the lubricant magnesium stearate, and optionally
(e) 0.1-3% by weight of the glidant colloidal silicon dioxide.

In a preferred embodiment of the present invention, the pharmaceutical composition comprises:
(a) 10-40% by weight of the drug substance (R,E)-N-(7-chloro-1-(1-(4-(dimethylamino)but-2-enoyl)azepan-3-yl)-1H-benzo[d]imidazol-2-yl)-2-methylisonicotinamide, calculated based on its free base and on its anhydrous basis, present as mono-mesylate trihydrate salt,
(b) 50-70% by weight of the fillers of mannitol and microcrystalline cellulose together,
(c) 3-8% by weight of the disintegrant crospovidone,
(d) 2-4% by weight of the lubricant magnesium stearate, and optionally
(e) 0.2-2% by weight of the glidant colloidal silicon dioxide.

In a more preferred embodiment of the present invention, the pharmaceutical composition comprises:
(a) 20-30% by weight of the drug substance (R,E)-N-(7-chloro-1-(1-(4-(dimethylamino)but-2-enoyl)azepan-3-yl)-1H-benzo[d]imidazol-2-yl)-2-methylisonicotinamide, calculated based on its free base and on its anhydrous basis, present as mono-mesylate trihydrate salt,
(b) 55-65% by weight of the fillers of mannitol and microcrystalline cellulose together,
(c) 4-7% by weight of the disintegrant crospovidone,
(d) 2-3% by weight of the lubricant magnesium stearate, and optionally
(e) 0.2-1% by weight of the glidant colloidal silicon dioxide.

Even more preferably, in said embodiments of the present invention, the pharmaceutical composition essentially consisting of, preferably consisting of:
(a) the drug substance (R,E)-N-(7-chloro-1-(1-(4-(dimethylamino)but-2-enoyl)azepan-3-yl)-1H-benzo[d]imidazol-2-yl)-2-methylisonicotinamide as mono-mesylate trihydrate salt,
(b) the fillers of mannitol and microcrystalline cellulose,
(c) the disintegrant crospovidone,
(d) a lubricant, preferably magnesium stearate,
(e) a glidant, preferably colloidal silicon dioxide, and
(f) a coating material, preferably a hypromellose-based coating material.

The term “essentially consisting of” indicates herein the tolerance of the presence of small amounts of other components which are present as undesired impurities or side products originating from the manufacturing process of said components or formed during the manufacturing process of the pharmaceutical dosage form, or as desired small-amount components. For example, the coating material may contain, in addition to hypromellose, some smaller amounts of compounds selected from the group of plasticizer(s) [e.g. polyethylene glycol (PEG) 4000], colorant(s) [e.g. iron oxide, red (E172), titanium dioxide (E171), iron oxide, black (E172)], anti-tack agent(s) [e.g. talc], and residual solvent(s) [e.g. water].

In a second aspect of the present invention, there is provided a process for the preparation of a pharmaceutical composition as defined by the first aspect of the present invention comprising the following steps:
(1) dry granulation of a blend composed of
   (a) the drug substance (R,E)-N-(7-chloro-1-(1-(4-(dimethylamino)but-2-enoyl)azepan-3-yl)-1H-benzo[d]imidazol-2-yl)-2-methylisonicotinamide, a pharmaceutically acceptable salt, hydrate, or salt hydrate thereof, preferably the mono-mesylate trihydrate salt thereof,
   (b) the filler microcrystalline cellulose, preferably of the quality 101,
   (c) the disintegrant crospovidone,
   (d) a lubricant, preferably magnesium stearate,
   and optionally
   (e) the filler mannitol, and optionally
   (f) a glidant, preferably colloidal silicon dioxide,
   to obtain granules;
(2) compression of the granules obtained by step (1) together with a blend composed of
   (g) the filler microcrystalline cellulose, preferably of the quality 102,
   (h) the disintegrant crospovidone,
   (i) a lubricant, preferably magnesium stearate,
   and optionally
   (j) the filler mannitol, and optionally
   (k) a glidant, preferably colloidal silicon dioxide,
   to obtain tablets;
wherein in either step (1) or step (2) the filler mannitol (component (e) or (j)) must be used;
and optionally
(3) film coating of the tablets obtained by step (2), preferably with coating suspension or solution composed of hypromellose.

Direct compression was found to be sub-optimal due to high level of sticking, capping and binding in dies with the compound of the present invention.

The advantage of the dry granulation process of the present invention is that wetting and drying steps can be avoided and that therefore the risk of solid phase conversions of the trihydrate of the mesylate salt of the compound of the present invention is minimized.

As an alternative embodiment of the second aspect of the present invention, there is provided a process for the preparation of a pharmaceutical composition as defined by the first aspect of the invention comprising the following steps:
(1) dry granulation of a blend composed of
   (a) the drug substance (R,E)-N-(7-chloro-1-(1-(4-(dimethylamino)but-2-enoyl)azepan-3-yl)-1H-benzo[d]imidazol-2-yl)-2-methylisonicotinamide, a pharmaceutically acceptable salt, hydrate, or salt hydrate thereof, preferably the mono-mesylate trihydrate salt thereof,
   (b) the filler microcrystalline cellulose, preferably of the quality PH101,
   (c) the disintegrant crospovidone,
   (d) a lubricant, preferably magnesium stearate,
   and optionally
   (e) the filler mannitol, and optionally
   (f) a glidant, preferably colloidal silicon dioxide,
   to obtain granules;
(2) filling of the granules obtained by step (1) together with a blend composed of
   (g) the filler microcrystalline cellulose, preferably of the quality PH102,
   (h) the disintegrant crospovidone,
   (i) a lubricant, preferably magnesium stearate,
   and optionally
   (j) the filler mannitol, and optionally
   (k) a glidant, preferably colloidal silicon dioxide,
   into capsules, preferably hard gelatin capsules;
wherein in either step (1) or step (2) the filler mannitol (component (e) or (j)) must be used.

Preferably, the dry granulation step (1) in said processes of the present invention comprises roller compaction with subsequent milling, said milling preferably comprising the use of screens with a screen size from 0.8 to 2.0 mm, preferably 0.8 mm, to obtain the granules.

Roller compaction provides the advantage of a mechanically gentler method compared to other dry granulation methods, e.g. slugging and may further minimize the risk of solid phase conversions of the trihydrate mesylate salt of the compound of the present invention.

The milling step is required to break the ribbons, sheets, flakes formed by the roller compaction step into granules of desired particle size, preferably smaller than 2 mm, more preferably smaller than 1 mm, even more preferably smaller than 0.8 mm.

As a third aspect of the present invention, there is provided a pharmaceutical tablet obtainable by the process as defined by the second aspect of the present invention.

As a fourth aspect of the present invention, there is provided a pharmaceutical capsule obtainable by the process as defined by the second aspect of the present invention.

### Examples

Hereinafter, the present invention is described in more details and specifically with reference to the examples, which however are not intended to limit the present invention.

Abbreviations used:

API active pharmaceutical ingredient

DS drug substance, synonymous for API

EGF816-AGA compound of formula (1) as mesylate salt trihydrate

FCT film coated tablet

IPC in process control

MS molecular size

TLC thin layer chromatography

XL cross-linked

### Example 1: Tablet Compositions

The following tables provide composition details of tablets in the dosage strengths 25, 50, and 200 mg.

TABLE 1-1
Composition of EGF816 25 mg FCT per unit and 10,000 tablets

| | | | Composition |
|---|---|---|---|
| | | Composition | (g) per |
| | | per | 10,000 |
| | | unit (mg) | tablets⁴ |
| | Component | 25 mg | 25 mg |
| Inner phase | EGF816-AGA¹ | 29.85⁶ | 298.5 |
| | Mannitol DC | 40.32 | 403.2 |
| | Avicel PH101² | 16.88 | 168.8 |
| | [microcrystalline cellulose | | |
| | PH101] | | |
| | Polyvinylpolypyrrolidon XL | 1.47 | 14.7 |
| | [crospovidone XL] | | |
| | Aerosil 200 [colloidal silicon | 0.12 | 1.2 |
| | dioxide] | | |
| | Magnesium stearate | 0.74 | 7.4 |
| Outer phase | Cellulose MK GR | 5.00 | 50.0 |
| | [microcrystalline cellulose | | |
| | PH102] | | |
| | Polyvinylpolypyrrolidon XL | 4.00 | 40.0 |
| | [crospovidone XL] | | |
| | Aerosil 200 [colloidal silicon | 0.37 | 3.7 |
| | dioxide] | | |
| | Magnesium stearate | 1.25 | 12.5 |
| TOTAL | | 100.0 | 1000 |
| Coating⁵ | Opadry I (hypromellose) | | |
| | Basic coating premix red | 0.4209 | 4.209 |
| | Basic coating premix white | 2.5338 | 25.338 |
| | Basic coating premix black | 0.0453 | 0.453 |
| | Purified water³ | qs | qs |
| TOTAL | | 103 | 1030.0 |

¹EGF816-AGA is a mesylate (methylsulphonate) trihydrate salt, this assumes a salt factor of 1.194 on an anhydrous basis. The actual DS quantity is to be adjusted for a content ≤99.5% or ≥100.5%.
²Excipient used as compensating material for API purity (Avicel PH101)
³Removed during processing
⁴Range 40,000 tablets to 250,000 tablets
⁵Coating as one batch or sub-batches according to the batch size and pan loading/availability
⁶Equivalent to 32.58 mg, assuming a salt factor including trihydrate of 1.303

TABLE 1-2
Composition of EGF816 50 mg FCT per unit and 10,000 tablets

| | | | Composition |
|---|---|---|---|
| | | Composition | (g) |
| | | per | per 10,000 |
| | | unit (mg) | tablets⁴ |
| | Component | 50 mg | 50 mg |
| Inner phase | EGF816-AGA¹ | 59.70⁶ | 597.0 |
| | Mannitol DC | 80.00 | 800.0 |
| | Avicel PH101² | 33.32 | 333.2 |
| | [microcrystalline cellulose | | |
| | PH101] | | |
| | Polyvinylpolypyrrolidon XL | 3.00 | 30.0 |
| | [crospovidone XL] | | |
| | Aerosil 200 [colloidal silicon | 0.24 | 2.4 |
| | dioxide] | | |
| | Magnesium stearate | 2.50 | 25.0 |
| Outer phase | Cellulose MK GR | 10.00 | 100.0 |
| | [microcrystalline cellulose | | |
| | PH102] | | |
| | Polyvinylpolypyrrolidon XL | 8.00 | 80.0 |
| | [crospovidone XL] | | |
| | Aerosil 200 [colloidal silicon | 0.74 | 7.4 |
| | dioxide] | | |
| | Magnesium stearate | 2.50 | 25.0 |
| TOTAL | | 200.0 | 2000 |
| Coating⁵ | Opadry I (hypromellose) | | |
| | Basic coating premix red | 0.8418 | 8.418 |
| | Basic coating premix white | 5.0676 | 50.676 |
| | Basic coating premix black | 0.0906 | 0.906 |
| | Purified water³ | qs | qs |
| TOTAL | | 206 | 2060.0 |

¹EGF816-AGA is a mesylate (methylsulphonate) trihydrate salt, this assumes a salt factor of 1.194 on an anhydrous basis. The actual DS quantity is to be adjusted for a content ≤99.5% or ≥100.5%.
²Excipient used as compensating material for API purity (Avicel PH101)
³Removed during processing
⁴Range 40,000 tablets to 250,000 tablets
⁵Coating as one batch or sub-batches according to the batch size and pan loading/availability
⁶Equivalent to 65.16 mg, assuming a salt factor including trihydrate of 1.303

TABLE 1-3
Composition of EGF816 200 mg FCT per unit and 10,000 tablets

| | | | Composition |
|---|---|---|---|
| | | Composition | (g) per |
| | | per | 10,000 |
| | | unit (mg) | tablets⁴ |
| | Component | 200 mg | 200 mg |
| Inner phase | EGF816-AGA¹ | 238.80⁶ | 2388.0 |
| | Avicel PH101² | 135.04 | 1350.4 |
| | [microcrystalline cellulose | | |
| | PH101] | | |
| | Polyvinylpolypyrrolidon XL | 11.76 | 117.6 |
| | [crospovidone XL] | | |
| | Aerosil 200 [colloidal silicon | 0.96 | 9.6 |
| | dioxide] | | |
| | Magnesium stearate | 5.92 | 59.2 |
| Outer phase | Mannitol DC | 322.56 | 3225.6 |
| | Cellulose MK GR | 40.00 | 400.0 |
| | [microcrystalline cellulose | | |
| | PH102] | | |
| | Polyvinylpolypyrrolidon XL | 32.00 | 320.0 |
| | [crospovidone XL] | | |
| | Aerosil 200 [colloidal silicon | 2.96 | 29.6 |
| | dioxide] | | |
| | Magnesium stearate | 10.00 | 100.0 |
| TOTAL | | 800.0 | 8000 |
| Coating⁵ | Opadry I (hypromellose) | | |
| | Basic coating premix red | 3.0866 | 30.866 |
| | Basic coating premix white | 18.5812 | 185.812 |
| | Basic coating premix black | 0.3322 | 3.322 |
| | Purified water³ | qs | qs |
| TOTAL | | 822.0 | 8220.0 |

¹EGF816-AGA is a mesylate (methylsulphonate) trihydrate salt, this assumes a salt factor of 1.194 on an anhydrous basis. EGF816-AGA is also a trihydrate therefore the actual DS quantity is to be adjusted for a content ≤99.5% or ≥100.5%.
²Excipient used as compensating material for API purity (Avicel PH101)
³Removed during processing
⁴Range 40,000 tablets to 150,000 tablets
⁵Coating as one batch or sub-batches according to the batch size and pan loading/availability
⁶Equivalent to 260.60 mg, assuming a salt factor including trihydrate of 1.303

### Example 2: Generic Description of the Tablet Manufacturing Process

Tablets of the compositions as indicated in example 1 are prepared as follows.

All ingredients of the internal phase except of magnesium stearate are screened through 0.8-1.2 mm (preferred settings 1.0 mm) using an oscillating mill (e.g. Frewitt Coni-Vitt-150 or Quadro Comil) and then loaded to a diffusion mixer(tumble)/bin blender, e.g. Bohle PM 400S, HF05. The mixture is blended with 17-20 rpm for 10 min.

Magnesium stearate is sieved by hand through 0.5-1.0 mm, preferred setting 0.8 mm, directly into the bin blender with the pre-blended ingredients. The mixture is blended with 17-20 rpm for further 2-3 min (lubrication).

The resulting lubricated blend is subjected to roller compaction using, e.g. the Bepex Pharmapaktor L-200/30, applying compaction forces of 10-35 kN and roller speed (revolution compaction roll) of 2-10 rpm.

The resulting ribbons are screened through 0.8 mm using, e.g. an oscillating mill (e.g. Frewitt Coni-Vitt-150 or Quadro Comil).

The resulting granules are blended together with the ingredients of the external phase. Again, first without magnesium stearate, with 17-20 rpm for 10 min, and then, after addition of the 0.8 mm screened magnesium stearate, with 17-20 rpm for further 2-3 min.

The resulting final blend is subjected to a compression rotary press (e.g. FETTE 1200i or Korsch XL400), using punches such as Euro B (max 19 mm) and Euro D (max 25 mm). Compression force settings, including optionally pre-compression forces (up to 20% of main compression force (MCF)), and adjusted to obtain tablets with the following hardness (IPC tests on core tablets):

| | | | | | Hardness: |
|---|---|---|---|---|---|
| | | | | | target |
| Dosage | Target | | | | (range of |
| strength | weight | | Diameter | Thickness | mean of 20 |
| [mg] | [mg] | Shape | [mm] | [mm] | tablets) [N] |
| 25 | 100 | round, | 6 | 3.3 | 60 (45-100) |
| | | biconvex | | | |
| 50 | 200 | round, | 8 | 3.7 | 80 (65-115) |
| | | biconvex | | | |
| 200 | 800 | ovaloid, | Length: 18 | 7.1 | 190 (165-250) |
| | | biconvex | Width: 7.1 | | |

All the resulting tablet cores were found to meet the following friability and disintegration time test criteria:
Friability (Ph.Eur., 20 tablets or at least 6.5 g of the tablets):
   no breakage, abrasion ≤0.8% after 500 drops.
Disintegration time (Ph.Eur., 6 units tested without disc, at water temperature 37° C.):
   ≤15 min.

The tablet cores are finally film coated using film coating perforated pans, e.g. Glatt GMPC II or Glatt GC 750 or 1000. For the aqueous film coating, the basic coating premix (Opadry I by Colorcon, hypromellose-based) is made up as a 15% w/w suspension and applied on a weight gain basis. The operational parameters are adjusted to obtain film coated tablets with the following characteristics:

| Dosage | Target average mass | | Thickness: target |
|---|---|---|---|
| strength | (tolerance | | (tolerance range) |
| [mg] | range) [mm] | Diameter range [mm] | [mm] |
| 25 | 103 (98.88-107.12) | 6.1-6.3 | 3.4 (3.2-3.6) |
| 50 | 206 (197.76-214.24) | 8.1-8.3 | 3.8 (3.6-4.0) |
| 200 | 822 (797.34-846.66) | Length: 18.1 | 7.2 (7.0-7.4) |
| | | Width: 7.2 | |

All the resulting film coated tablets were found to meet the following disintegration time test criteria:
Disintegration time (Ph.Eur., 6 units tested without disc, at water temperature 37° C.):
   ≤15 min.

### Example 3: Analytical Results from Large Scale Tablet Batches

The process of example 2 was used to prepare tablets with the compositions as outlined in example 1 on large scale. The following tables provide the analytical results of IPC tests as well as Processibility and Purity tests with the final product.

TABLE 3-1
Process parameters and IPC values for large scale tablet example batches

| | Example batch | | | |
|---|---|---|---|---|
| | 3-1 | 3-2 | 3-3 | 3-4 |
| Strength | 25 mg | 25 mg | 200 mg | 200 mg |
| Batch size (ST) | 45,000 | 150,000 | 45,000 | 65,000 |
| Batch size (kg) | 4.499 | 15.002 | 31.996 | 51.998 |
| Thickness (mm) | 3.3-3.4 | 3.3-3.4 | 7.1-7.2 | 7.2-7.3 |
| Average Pre-compression | 0.5 | 0.0 | 0.1 | 0.5 |
| force (kN) | | | | |
| Average Compression force | 4.5 | 2.6 | 11.0 | 7.0 |
| (kN) | | | | |
| Mean hardness (N) | 82 (65-97) | 102 (82-114) | 224 (205-249) | 253 (269-253) |
| Friability (%) | 0.03-0.09 | 0 | 0-0.1 | 0-0.02 |
| Disintegration time (DT) | 6.44-8.06 | 3.43-8.58 | 6.20-10.05 | 5.02-12.45 |
| (min) | | | | |
| Carr's Index | 19.40 | 22.06 | 21.13 | 20.29 |
| (granulate inner phase) | | | | |
| Carr's Index (final blend) | 14.71 | 18.57 | 14.29 | 16.67 |
| Hausner ratio | 1.24 | 1.28 | 1.27 | 1.26 |
| (granulate inner phase) | | | | |
| Hausner ratio (final blend) | 1.17 | 1.23 | 1.17 | 1.20 |

The data demonstrates that final blends of the compositions of the present invention are of good pharmaceutical processablility (good flow properties) and possess good compression characteristics and that the resulting tablets are quickly disintegratable.

TABLE 3-2
Particle size distribution (PSD) of final blends of large scale example
batches

Example batch
Sieve aperture (μm)

| | 3-1 | 3-2 | 3-3 | 3-4 |
|---|---|---|---|---|
| Fines | 10.9 | 15.2 | 10.7 | 12.4 |
| 63 | 9.1 | 8.4 | 7.1 | 6.6 |
| 90 | 8.7 | 8.4 | 7.9 | 11.2 |
| 125 | 10.1 | 9.0 | 16.8 | 14.8 |
| 180 | 9.7 | 10.0 | 15.8 | 13.6 |
| 250 | 11.3 | 10.4 | 13.2 | 12.2 |
| 355 | 17.1 | 17.4 | 15.0 | 14.8 |
| 500 | 20.7 | 19.8 | 13.0 | 13.4 |
| 710 | 2.2 | 1.4 | 0.6 | 1.0 |
| 1000 | 0 | 0 | 0 | 0 |

The PSD data demonstrates that the compositions and the process of the present invention consistently provide blends free of large amounts of fines and coarse material, an indication of good pharmaceutical processability.

TABLE 3-3
Quality control (QC) results for final tablets of large scale example batches

| | Example batch | | | |
|---|---|---|---|---|
| | 3-1 | 3-2 | 3-3 | 3-4 |
| Water (%) | 4.614 | 4.0834 | 4.5174 | 4.1781 |
| Assay (%) | 101.8 | 100.6 | 102.4 | 102.3 |
| Content uniformity (CU) | 3.4 | 6.9 | 4.7 | 2.7 |
| Total Impurities (%) | 0.2 | <0.1 | 0.2 | <0.1 |

These results demonstrate that tablets made of the compositions of the present invention with the process of the present invention are of high purity and high content uniformity.

Dissolution testing was conducted on the example batches using 0.1M HCL, paddle apparatus at 50 rpm, 37° C. For all examples batches, 100% dissolution was observed within 15-20 min indicating complete and fast drug release.

In long term storage tests at 25° C./60% RH (relative humidity), no significant change or trend with respect to degradation products or impurities was observed and even the data from accelerated conditions (40° C./75% RH) did not exceed the specification for degradation products (≤0.5%). Therefore, the compositions as described in example 1 and processed according to example 2 resulted in stable tablets suitable for human use.

### Example 4: Test Batches

According to the process of example 2, the following compositions were processed to 200 mg dosage strength tablets and the dissolution and compressability were studied.

TABLE 4-1
Test batch compositions

| | 4-1 | 4-2 | 4-3 |
|---|---|---|---|
| Test batches | “SSG” | “PVP-XL” | “L-HPC” |
| Materials | (%) | (%) | (%) |
| EGF816-AGA | 33.24 | 33.24 | 33.24 |
| Avicel PH101 | 13.49 | 13.49 | 13.49 |
| Mannitol DC | 40.32 | 40.32 | 40.32 |
| Sodium Starch Glycolate | 1.47 | — | — |
| Crospovidone | — | 1.47 | — |
| HP Cellulose Low substituted | — | — | 1.47 |
| Aerosil 200 | 0.12 | 0.12 | 0.12 |
| Magnesium stearate | 0.74 | 0.74 | 0.74 |
| Total dry blend (for compaction) | 89.38 | 89.38 | 89.38 |
| Cellulose MK GR | 5.00 | 5.00 | 5.00 |
| Crospovidone | — | 4.00 | — |
| Sodium Starch Glycolate | 4.00 | — | — |
| HP Cellulose Low substituted | — | — | 4.00 |
| Aerosil 200 | 0.37 | 0.37 | 0.37 |
| Magnesium stearate | 1.25 | 1.25 | 1.25 |
| Total final blend (%) | 100.00 | 100.00 | 100.00 |

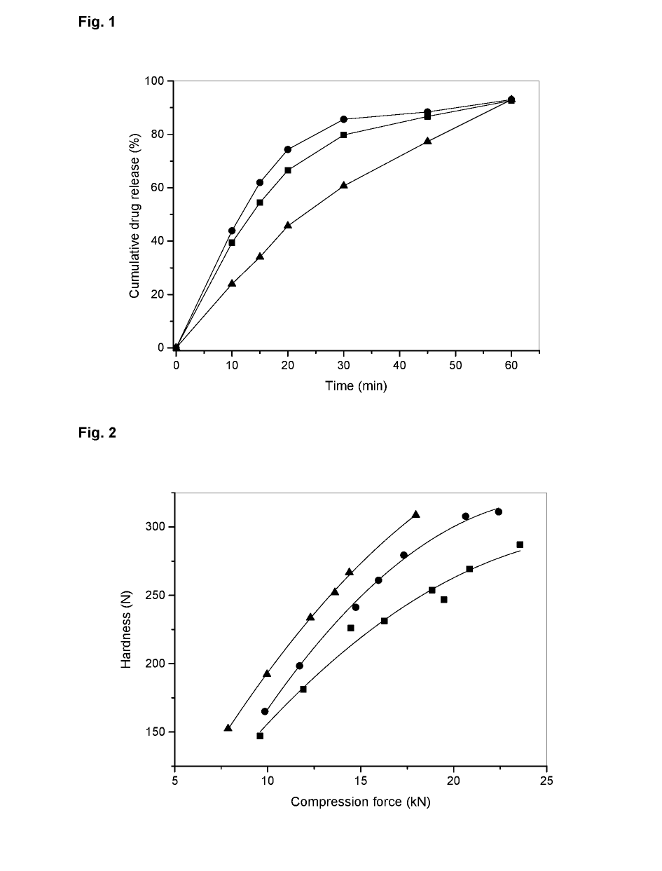

FIG. 1 shows the results of the dissolution test which was performed at pH 6.8, using the paddle method (50 rpm, 900 mL) and demonstrates that the composition with low-substituted hydroxylpropyl cellulose (L-HPC) results in tablets which only slowly release the drug substance. The order of dissolution rate is PVP-XL>SSG>L-HPC (>CMC-XL)*. (* not shown in graph, comparable tablet batches with CMC-XL showed drug release of only about 70%.)

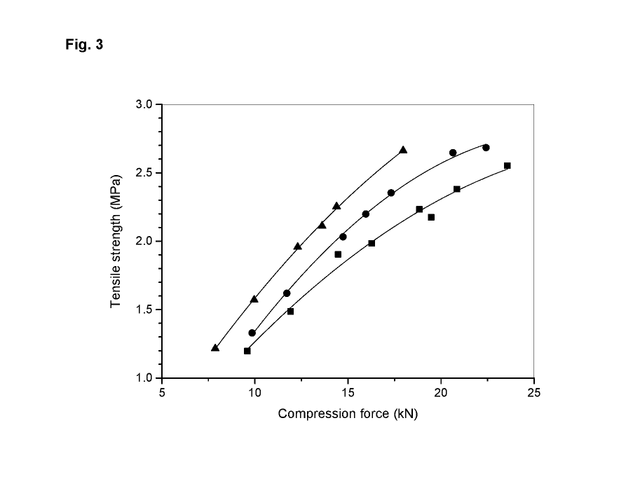

FIG. 2 and FIG. 3 show the results of the compressibility tests which were performed using a Fette P1200-Euro B with the punches 18×7.1 mm at a machine speed of 20 rpm. The compression force is the mean value measured for the upper and lower punch. The tensile strength is calculated taking into account the hardness and the thickness of the resulting tablets. The results demonstrate that the order of compressibility is L-HPC>PVP-XL (>CMC-XL)*>SSG. (* not shown in graph, comparable tablet batches with CMC-XL showed tensile strength versus compression curves in between of the curves for PVP-XL and SSG.)

Dissolution and compression tests, together, demonstrate the superior characteristics of compositions comprising crospovidone (PVP-XL) compared to compositions with other disintegrants.

### Example 5: Preparation of EGF816 Mesylate Trihydrate

### Example 5.1 Preparation of (R,E)-N-(7-chloro-1-(1-(4-(dimethylamino)but-2-enoyl)azepan-3-yl)-1H-benzo[d]imidazol-2-yl)-2-methylisonicotinamide

### Intermediate 15

### (S)-tert-butyl 3-(2-amino-5-methyl-1H-benzo[d]imidazol-1-yl) piperidine-1-carboxylate

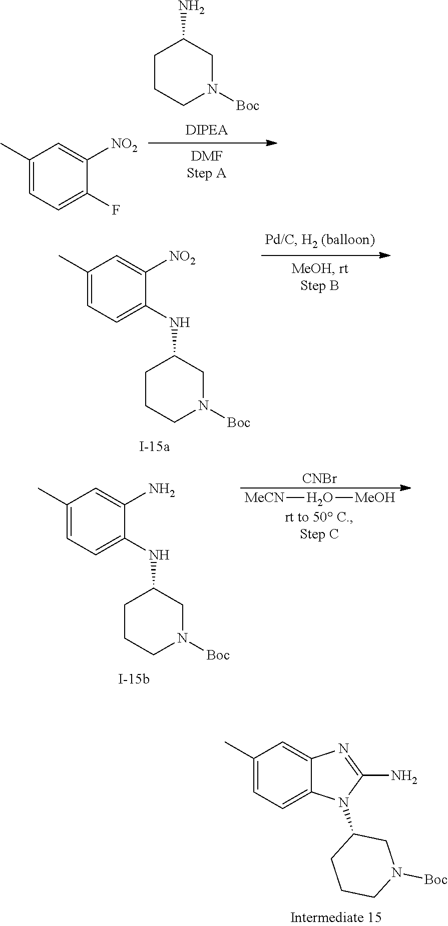

Step A:

A stirred solution of (S)-tert-butyl 3-aminopiperidine-1-carboxylate (0.500 g, 2.49 mmol), 1-fluoro-4-methyl-2-nitrobenzene (0.387 g, 2.49 mmol) and N,N-diisopropylethylamine (0.482 g, 3.74 mmol) in DMF under argon was heated to 110° C. for 6h (reaction completion monitored by TLC). The mixture was diluted with water and extracted with EtOAc (3×100 mL). The combined organic layers were dried over anhydrous Na₂SO₄ and concentrated under reduced pressure to afford (S)-tert-butyl 3-((4-methyl-2-nitrophenyl) amino) piperidine-1-carboxylate (I-15a). MS calculated for C₁₇H₂₄N₃O₄ (M−H⁻) 334.18, found 334.0.

Step B:

To a stirred solution of I-15a (0.550 g, 1.64 mmol) in MeOH (35 mL) was added Pd/C (0.090 g) and the mixture was stirred at room temperature under hydrogen atmosphere (balloon) for 2h (reaction completion monitored by TLC). The mixture was filtered through Celite, washed with MeOH and concentrated under reduced pressure to afford (S)-tert-butyl 3-((2-amino-4-methylphenyl)amino)piperidine-1-carboxylate (I-15b). MS calculated for C₁₇H₂₈N₃O₂ (M+H⁺) 306.22, found 306.2.

Step C:

To a stirred solution of (S)-tert-butyl 3-((2-amino-4-methylphenyl)amino)piperidine-1-carboxylate (I-15b) (0.500 g, 1.63 mmol) in MeOH (20 mL) was added a solution of cyanogen bromide (0.208 g, 1.96 mmol) in 1:2 MeCN:H₂O (20 mL) for a period of 5 min. The mixture was heated to 50° C. for 2h (reaction completion monitored by TLC), cooled to 00° C. and pH was adjusted to 10 by adding aqueous Na₂CO₃ solution. The mixture was stirred for 30 min at room temperature, the resulting solid was collected and dried under vacuum to afford the title compound (Intermediate 15). ¹H-NMR (400 MHz, CDCl₃): ∂7.24 (s, 1H), 7.17 (d, J=7.6 Hz, 1H), 6.85 (d, J=8 Hz, 1H), 4.64 (br s, 2H), 4.17 (t, J=14.8 Hz, 2H), 3.99-3.93 (m, 1H), 3.32 (d, J=11.6 Hz, 1H), 2.79 (t, J=12.4 Hz, 1H), 2.41 (s, 3H), 2.38-2.37 (m, 1H), 2.34 (d, J=3.2 Hz, 1H), 1.91 (d, J=13.6 Hz, 3H), 1.69-1.61 (m, 1H), 1.47 (s, 9H); MS calculated for C₁₈H₂₇N₄O₂ (M+H⁺) 331.21, found 331.0.

### Intermediate 26

### (R)-tert-butyl 3-(2-amino-7-chloro-1H-benzo[d]imidazol-1-yl)azepane-1-carboxylate

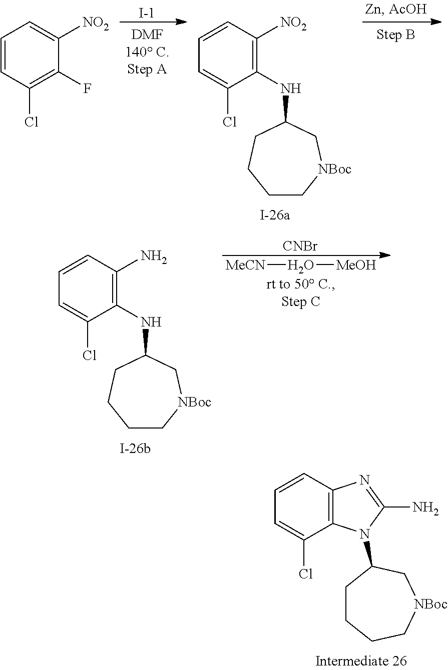

Step A:

(R)-tert-butyl 3-((2-chloro-6-nitrophenyl)amino)azepane-1-carboxylate (I-26a) was prepared following procedures analogous to 1-15, Step A, using the appropriate starting materials. ¹H-NMR (400 MHz, CDCl₃): ∂ 8.00-7.91 (m, 1H), 7.58-7.49 (m, 1H), 7.02-6.51 (m, 2H), 4.31-4.03 (m, 1H), 3.84-2.98 (m, 4H), 1.98-1.60 (m, 5H), 1.46-1.39 (m, 10H); MS calculated for C₁₇H₂₅ClN₃O₄(M+H⁺) 370.15, found 370.10.

Step B:

A mixture of I-26a (7.5 g, 19.5 mmol) and Zn (12.8 mg, 195 mmol) in AcOH (22 mL) was stirred at room temperature for 2 h. The reaction was basified with saturated aqueous Na₂CO₃ solution, filtered, and extracted with EtOAc (3×80 mL). The combined organic phase was washed with brine, dried with Na₂SO₄ and concentrated in vacuum to afford (R)-tert-butyl 3-((2-amino-6-chlorophenyl)amino)azepane-1-carboxylate (I-26b). MS calculated for C₁₇H₂₇ClN₃O₂(M+H⁺) 340.17, found 340.10. The crude was used in the next step without further purification.

Step C:

The title compound (Intermediate 26) was prepared from I-26b following procedures analogous to I-15, Step C. ¹H-NMR (400 MHz, CDCl₃): ∂ 7.34-7.26 (m, 1H), 7.04-6.97 (m, 2H), 6.05-5.85 (m, 1H), 5.84-5.72 (m, 1H), 5.50-5.37 (m, 0.5H), 5.10-4.80 (m, 0.5H), 4.41-4.23 (m, 1H), 4.09-3.96 (m, 0.5H), 3.94-3.81 (m, 1H), 3.76-3.57 (m, 1H), 3.22-3.14 (m, 0.5H), 2.84-2.63 (m, 1H), 2.34-2.17 (m, 1H), 2.07-1.84 (m, 1H), 1.82-1.64 (m, 2H), 1.53 (s, 9H), 1.48-1.37 (m, 1H); MS calculated for C₁₈H₂₆ClN₄O₂(M+H⁺) 365.17, found 365.10.

### Intermediate 27

### (R)—N-(1-(azepan-3-yl)-7-chloro-1H-benzo[d]imidazol-2-yl)-2-methylisonicotinamide hydrochloride

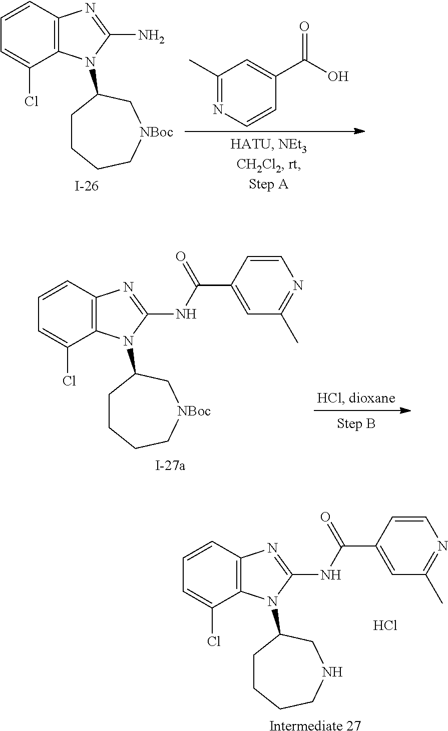

Step A:

A mixture of 2-methylisonicotinic acid (3.371 g, 24.6 mmol) and 2-(7-aza-1H-benzotriazole-1-yl)-1,1,3,3-tetramethyluronium hexafluorophosphate (9.345 g, 24.6 mmol) in CH₂Cl₂ (120 ml) was treated at room temperature with NEt₃ (4.1 mL, 29.4 mmol). The reaction was stirred for 1 hour before it was slowly added into a CH₂Cl₂ solution (45 ml) of I-26 (5.98 g, 16.4 mmol). Ten minutes later, more NEt₃ (4.1 mL, 29.4 mmol) was added and the mixture stirred for 2 h. The mixture was then diluted with CH₂Cl₂ (240 mL), washed with H₂O (2×80 mL), saturated aqueous NaHCO₃ solution (70 mL), and brine (70 mL). The organic phase was dried with Na₂SO₄, and concentrated under reduced pressure. The crude material was purified by column chromatography (55% EtOAc/hexanes) to afford (R)-tert-butyl 3-(7-chloro-2-(2-methylisonicotinamide)-1H-benzo[d]imidazol-1-yl)azepane-1-carboxylate (I-27a) as a light yellow foam. ¹H-NMR (400 MHz, CDCl₃): ∂ 12.81 (br s, 1H), 8.65-8.62 (m, 1H), 7.95-7.85 (m, 2H), 7.27-7.11 (m, 3H), 5.64-5.51 (m, 1H), 4.56-4.44 (m, 1H), 4.07-3.92 (m, 1H), 3.79-3.71 (m, 0.5H), 3.41-3.35 (m, 0.5H), 3.29-3.23 (m, 1H), 2.71-2.59 (m, 1H), 2.65 (s, 3H), 2.22-2.00 (m, 3H), 1.93-1.80 (m, 1H), 1.51-1.45 (m, 1H), 1.50 (s, 3.5H), 1.41 (s, 5.5H); MS calculated for C₂₅H₃₁ClN₅O₃(M+H⁺) 484.20, found 484.20.

Step B:

A solution of I-27a (8.62 g, 16.4 mmol) in MeOH (67 mL) was treated with HCl in dioxane (4M, 67 mL) and the mixture was stirred at room temperature for 7 h. The mixture was then concentrated under reduced pressure to afford the title compound (Intermediate 27). The product was used in the next step without further purification. A sample was treated with 1M NaOH, extracted with EtOAc, dried with Na₂SO₄ and concentrated under reduced pressure to afford 1-27 as a free base. ¹H-NMR (400 MHz, CD₃CN): ∂ 8.49 (d, J=5.0 Hz, 1H), 7.81 (s, 1H), 7.72 (d, J=4.8 Hz, 1H), 7.50 (brd, J=7.52 Hz, 1H), 7.16-7.09 (m, 2H), 5.66-5.59 (m, 1H), 3.77 (dd, J=6.54, 14.3 Hz, 1H), 3.18 (dd, J=5.3, 14.3 Hz, 1H), 3.05-2.98 (m, 1H), 2.76-2.69 (m, 1H), 2.63-2.53 (m, 1H), 2.47 (s, 3H), 2.10-2.03 (m, 1H), 1.96-1.93 (m, 2H), 1.86-1.75 (m, 2H), 1.61-1.54 (m, 2H); MS calculated for C₂₀H₂₃ClN₅O (M+H⁺) 384.15, found 384.20.

### (R,E)-N-(7-chloro-1-(1-(4-(dimethylamino)but-2-enoyl)azepan-3-yl)-1H-benzo[d]imidazol-2-yl)-2-methylisonicotinamide

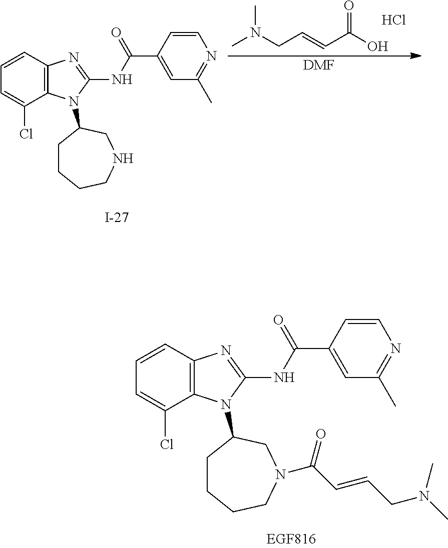

A mixture of (E)-4-(dimethylamino)but-2-enoic acid hydrochloride (58 mg, 0.35 mmol) and 1-ethyl-3-(3-dimethylaminopropyl)carbodiimide hydrochloride (67 mg, 0.35 mmol) in DMF (2 mL) was treated with hydroxybenzotriazole (54 mg, 0.35 mmol) and stirred at room temperature for 1 h. The resulting mixture was added to a solution of 1-27 (100 mg, 0.22 mmol) in DMF (2 mL). Triethylamine (199 mg, 1.97 mmol) was then added and the mixture was stirred for 5 days. Water (2 mL) was added and the mixture was concentrated under reduced pressure. The residue was diluted with 1N NaOH (20 mL) and extracted with EtOAc (3×50 mL). The combined organic layers were washed with water (50 mL) and brine (2×50 mL), dried over Na₂SO₄, and concentrated under reduced pressure. The crude was purified by column chromatography (9:1:0.175N CH₂Cl₂/MeOH/NH₃ in CH₂Cl₂, 0% to 100%) to afford the title compound. ¹H NMR (400 MHz, DMSO-d₆) ∂ 8.59 (d, J=4.8 Hz, 1H), 7.89 (s, 1H), 7.79 (d, J=4.8 Hz, 1H), 7.60 (d, J=7.5 Hz, 1H), 7.30-7.22 (m, 2H), 6.71-6.65 (m, 1H), 6.57-6.54 (m, 1H), 5.54 (br. s, 1H), 4.54 (br. s, 1H), 4.20 (br s, 1H), 3.95 (br s, 1H), 3.48 (br s, 1H), 2.98 (br s, 2H), 2.72 (d, J=12.0 Hz, 1H), 2.58 (s, 3H), 2.14 (brs, 6H), 2.05 (d, J=6.7 Hz, 3H), 1.88 (br s, 1H), 1.46 (d, J=11.3 Hz, 1H); MS calculated for C₂₆H₃₂ClN₆O₂(M+H⁺) 495.22, found 495.10. Melting point (114.6° C.).

### Example 5.2 Preparation of Crystalline Mesylate Form B (Mesylate Trihydrate Form)

(R,E)-N-(7-chloro-1-(1-(4-(dimethylamino)but-2-enoyl)azepan-3-yl)-1H-benzo[d]imidazol-2-yl)-2-methylisonicotinamide as obtained in Example 5.1 (1.0 g) was dissolved in acetone (30 mL) by heating to 55° C. to form a solution. Methanesulfonic acid (325 μL) was added to acetone (50 mL), and the methanesulfonic acid/acetone (22.2 mL) was added to the solution at 0.05 ml/min. Following precipitation, the resulting suspension was cooled to room temperature at 0.5° C./min, and crystals were collected by filtration, and dried for 4 hours at 40° C. under vacuum. The collected crystals (300 mg) were suspended in acetone/H₂O (6 mL; v/v=95/5) by heating to 50° C. The suspension was kept slurrying for 16 hours, and cooled to room temperature at 0.5° C./min. The crystal was collected by filtration and dried for 4 hours at 40° C. under vacuum.

The structure of (R,E)-N-(7-chloro-1-(1-(4-(dimethylamino)but-2-enoyl)azepan-3-yl)-1H-benzo[d]imidazol-2-yl)-2-methylisonicotinamide mesylate was confirmed by Differential Scanning Calorimetry, X-Ray Powder Diffraction, and Elemental Analyses. Melting point (170.1° C.). Theoretical calculated: % C (54.8); % H (5.9); % N (14.2); % O (13.5); % S (5.4); and % Cl (6.0); C:N ratio: 3.86. Found: % C (52.0); % H (5.8); % N (13.3); % Cl (5.9); C:N ratio: 3.91. Stoichiometry: 1.01.

In addition, crystalline mesylate form B was prepared by suspending 300 mg of crystalline mesylate form A (see example 5.3) in 6 mL of acetone/H2O (v/v=95/5) by heating to 50° C. The suspension was kept slurrying for 16 hours, and then the suspension was allowed to cool to room temperature at 0.5° C./min. The crystal was collected by filtration and afterwards dried for 4 hours at 40° C. under vacuum.

### Example 5.3 Crystalline Mesylate Form A (Mesylate Monohydrate Form)

5.0 mL of dried acetone and 800 mg of mesylate form B (mesylate trihydrate form) as obtained in example 3 were added into a glass vial. The suspension was heated to 55° C. for 5 hours. DSC was checked to see if the transformation was complete. Another 800 mg of the mesylate form B was converted to mesylate form A with the same method, the only difference was that the suspension was allowed to equilibrate at 20° C. (the ambient temperature in the lab), overnight.

In addition, crystalline mesylate form A was prepared by dissolving 1.0 g of free form A (see example 5.4) in 30 mL of acetone by heating to 55° C. 325 μL of methansulfonic acid was added to 50 mL of acetone and then 22.2 mL of methansulfonic acid acetone was added to free form solution at 0.05 ml/min. Precipitation was formed during the addition of methansulfonic acid, and the suspension was allowed to cool to room temperature at 0.5° C./min. The crystal was collected by filtration and afterwards dried for 4 hours at 40° C. under vacuum.

### Example 5.4 Preparation of Crystalline Free Form A (Anhydrous Form)

750 mg of EGFRi HCl salt form (purity: 99%) were dissolved in 15 mL of mixed solvent (EtOH/H2O, v/v=1/9) by heating to 60° C. 7.42 mL sodium hydroxide (0.2 mol/L in EtOH/H2O, v/v=1/9) was added to the HCl salt form EtOH/H2O solution at 0.05 ml/min. Precipitation was formed during the addition of sodium hydroxide, and the suspension was allowed to cool to room temperature at 0.5° C./min. The crystal was collected by filtration and afterwards dried for 4 hours at 40° C. under vacuum.

### Example 6: In Vivo Pharmacokinetic (PK) Performance

Non-small cell lung cancer patients with EGFR T790M mutation were enrolled in a clinical study and received escalating doses of EGF816 ranging from 100 mg to 225 mg daily oral doses by using the tablets as described in example 1. Steady state EGF816 PK parameters (Cycle 1 Day 15) for the tablets (n=23) are displayed in the Table 6-1.

TABLE 6-1
Steady state EGF816 PK parameters for the tablet formulation of example 1

| Treatment | Tmax | Cmax | AUClast | AUCtau | Racc | CLss/F | T½ |
|---|---|---|---|---|---|---|---|
| group | (h)* | (ng/mL)** | (h · ng/mL)** | (h · ng/mL)** | AUCtau** | (mL/min)** | (h)** |
| 100 mg qd | 6 (2-24) | 519 (46) | 9052 (36) | NA | NA | NA | 14 (***) |
| (N = 3) | | | | | | | |
| 150 mg qd | 5 (2-7) | 675 (46) | 10312 (41) | 10482 (44) | 1.5 (35) | 238 (38) | 17 (8) |
| (N = 6) | | | | | | | |
| 200 mg qd | 3 (2-8) | 939 (35) | 13534 (34) | 13486 (37) | 1.4 (46) | 247 (58) | 13 (14) |
| (N = 7) | | | | | | | |
| 225 mg qd | 3 (2-6) | 1272 (31) | 12147 (47) | 16882 (63) | 1.6 (40) | 222 (37) | 10 (34) |
| (N = 7) | | | | | | | |

*Tmax was expressed as median (minimum-maximum)
**Other PK parameters were expressed as geometric mean (CV % of mean)
(***) Only one value calculated. Thus, no CV % of mean.
NA = Not available based on PK analysis with the Phoenix 6.4 (Pharsight)
PK parameters were generated for patients with more than 4 data-points

1. A pharmaceutical composition comprising
(a) the drug substance (R,E)-N-(7-chloro-1-(1-(4-(dimethylamino)but-2-enoyl)azepan-3-yl)-1H-benzo[d]imidazol-2-yl)-2-methylisonicotinamide, a pharmaceutically acceptable salt, thereof,
(b) the fillers mannitol and microcrystalline cellulose,
(c) the disintegrant crospovidone, and
(d) a lubricant.

2. The pharmaceutical composition according to claim 1, wherein said drug substance is present as mono-mesylate trihydrate salt.

3. The pharmaceutical composition according to claim 2, wherein said drug substance is present, calculated based on its free base and on an anhydrous basis, is present from 5 to 50%, by weight based on the total weight of said pharmaceutical composition.

4. The pharmaceutical composition according to claim 3, wherein said fillers together are present from 20 to 90% by weight based on the total weight of said pharmaceutical composition.

5. The pharmaceutical composition according to claim 4, wherein the fillers mannitol and microcrystalline cellulose are present in a ratio of from 3:1 to 1:1 (weight of mannitol:weight of microcrystalline cellulose).

6. The pharmaceutical composition according to claim 5, wherein the disintegrant is present from 2 to 10%, by weight based on the total weight of said pharmaceutical composition.

7. The pharmaceutical composition according to claim 6, wherein the lubricant is calcium or magnesium stearate.

8. The pharmaceutical composition according to claim 7, wherein said lubricant is present in from 1 to 5% by weight based on the total weight of said pharmaceutical composition.

9. The pharmaceutical composition according to claim 8, wherein said pharmaceutical composition is a capsule.

10. The pharmaceutical composition according to claim 8, wherein said pharmaceutical dosage form is a tablet and the tablet is coated with a film comprising hypromellose.

11. The pharmaceutical composition according to claim 10, wherein said pharmaceutical dosage form comprises a drug substance dose selected from 25, 50, 75, and 100 mg of the drug substance referred to as its free base and in its anhydrous form.

12. The pharmaceutical composition according to claim 1 comprising:
(a) 5-50% by weight of the drug substance (R,E)-N-(7-chloro-1-(1-(4-(dimethylamino)but-2-enoyl)azepan-3-yl)-1H-benzo[d]imidazol-2-yl)-2-methylisonicotinamide, calculated based on its free base and on its anhydrous basis, present as mono-mesylate trihydrate salt,
(b) 20-90% by weight of the fillers of mannitol and microcrystalline cellulose together,
(c) 2-10% by weight of the disintegrant crospovidone,
(d) 1-5% by weight of the lubricant magnesium stearate, and optionally
(e) 0.1-3% by weight of the glidant colloidal silicon dioxide.

13. The pharmaceutical composition according to claim 1 comprising:
(a) 10-40% by weight of the drug substance (R,E)-N-(7-chloro-1-(1-(4-(dimethylamino)but-2-enoyl)azepan-3-yl)-1H-benzo[d]imidazol-2-yl)-2-methylisonicotinamide, calculated based on its free base and on its anhydrous basis, present as mono-mesylate trihydrate salt,
(b) 50-70% by weight of the fillers of mannitol and microcrystalline cellulose together,
(c) 3-8% by weight of the disintegrant crospovidone,
(d) 2-4% by weight of the lubricant magnesium stearate, and optionally
(e) 0.2-2% by weight of the glidant colloidal silicon dioxide.

14. The pharmaceutical composition according to claim 1 comprising:
(a) 20-30% by weight of the drug substance (R,E)-N-(7-chloro-1-(1-(4-(dimethylamino)but-2-enoyl)azepan-3-yl)-1H-benzo[d]imidazol-2-yl)-2-methylisonicotinamide, calculated based on its free base and on its anhydrous basis, present as mono-mesylate trihydrate salt,
(b) 55-65% by weight of the fillers of mannitol and microcrystalline cellulose together,
(c) 4-7% by weight of the disintegrant crospovidone,
(d) 2-3% by weight of the lubricant magnesium stearate, and optionally
(e) 0.2-1% by weight of the glidant colloidal silicon dioxide.

15. (canceled)

16. The pharmaceutical composition according to claim 14, wherein the glidant is colloidal silicon dioxide, preferably said colloidal silicon dioxide has a specific surface area of 200 m²/g.

17. A process for the preparation of a pharmaceutical composition according to claim 1 comprising the following steps:
(1) dry granulation of a blend composed of
(a) the drug substance (R,E)-N-(7-chloro-1-(1-(4-(dimethylamino)but-2-enoyl)azepan-3-yl)-1H-benzo[d]imidazol-2-yl)-2-methylisonicotinamide, a pharmaceutically acceptable salt, hydrate, or salt hydrate thereof, preferably the mono-mesylate trihydrate salt thereof,
(b) the filler microcrystalline cellulose, preferably of the quality 101,
(c) the disintegrant crospovidone,
(d) a lubricant, preferably magnesium stearate,
and optionally
(e) the filler mannitol, and optionally
(f) a glidant, preferably colloidal silicon dioxide,
to obtain granules;
(2) compression of the granules obtained by step (1) together with a blend composed of
(g) the filler microcrystalline cellulose, preferably of the quality 102,
(h) the disintegrant crospovidone,
(i) a lubricant, preferably magnesium stearate,
and optionally
(j) the filler mannitol, and optionally
(k) a glidant, preferably colloidal silicon dioxide,
to obtain tablets;
wherein in either step (1) or step (2) the filler mannitol (component (e) or (j)) must be used;
and optionally
(3) film coating of the tablets obtained by step (2), preferably with coating suspension or solution composed of hypromellose.

18. A process for the preparation of a pharmaceutical composition according to claim 1 comprising the following steps:
(1) dry granulation of a blend composed of
(a) the drug substance (R,E)-N-(7-chloro-1-(1-(4-(dimethylamino)but-2-enoyl)azepan-3-yl)-1H-benzo[d]imidazol-2-yl)-2-methylisonicotinamide, a pharmaceutically acceptable salt, hydrate, or salt hydrate thereof, preferably the mono-mesylate trihydrate salt thereof,
(b) the filler microcrystalline cellulose, preferably of the quality PH101,
(c) the disintegrant crospovidone,
(d) a lubricant, preferably magnesium stearate,
and optionally
(e) the filler mannitol, and optionally
(f) a glidant, preferably colloidal silicon dioxide,
to obtain granules;
(2) filling of the granules obtained by step (1) together with a blend composed of
(g) the filler microcrystalline cellulose, preferably of the quality PH102,
(h) the disintegrant crospovidone,
(i) a lubricant, preferably magnesium stearate,
and optionally
(j) the filler mannitol, and optionally
(k) a glidant, preferably colloidal silicon dioxide,
into capsules, preferably hard gelatin capsules;
wherein in either step (1) or step (2) the filler mannitol (component (e) or (j)) must be used.

19. The process according to claim 17, wherein the dry granulation step (1) comprises roller compaction with subsequent milling with a screen size from 0.8 to 2.0 mm to obtain the granules.

20. A pharmaceutical tablet obtained by the process of claim 19.

21. A pharmaceutical capsule obtained by the process of claim 18, wherein the dry granulation step (1) comprises roller compaction with subsequent milling with a screen size from 0.8 to 2.0 mm to obtain the granules.
